(19) United States
(12) Patent Application Publication
(10) Pub. No.: US 2024/0336983 A1
(43) Pub. Date: Oct. 10, 2024

(54) PSEUDOMONAS SYRINGAE PV. TABACI-RESISTANT SHORT WZ INTROGRESSION SEGMENT, USE, AND SCREENING METHOD THEREOF

(71) Applicant: Yunnan Academy Of Tobacco Agricultural Sciences, Kunming (CN)

(72) Inventor: Yong LIU, Kunming (CN)

(73) Assignee: Yunnan Academy Of Tobacco Agricultural Sciences, Kunming (CN)

(21) Appl. No.: 18/354,694

(22) Filed: Jul. 19, 2023

(30) Foreign Application Priority Data
Apr. 4, 2023 (CN) 202310353049.4

(51) Int. Cl. C12Q 1/6895; A01H 1/04

(52) U.S. Cl. CPC C12Q 1/6895; A01H 1/04; C12Q 2600/13

(57) ABSTRACT

A Pseudomonas syringae pv. tabaci-resistant short WZ introgression segment and use thereof are provided. Compared with a long WZ introgression segment, the short WZ introgression segment is reduced by a drag gene component of at least 500 Kb. The short WZ introgression segment can be used for selective breeding of a Pseudomonas syringae pv. tabaci-resistant N. tabacum variety without obvious yield and quality disadvantages, which has promising application prospects. A simple screening method for an N. tabacum plant with the short WZ segment is also provided, where a homozygous N. tabacum plant with a long WZ introgression segment is hybridized with an N. tabacum plant without a long WZ introgression segment to quickly and efficiently obtain an N. tabacum plant with the short WZ segment, which provides an experimental basis for the acquisition of the Pseudomonas syringae pv. tabaci-resistant short WZ introgression segment.

### CROSS REFERENCE TO THE RELATED APPLICATIONS

This application is based upon and claims priority to Chinese Patent Application No. 2023103530494, filed on Apr. 4, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBDHHY008-PKG_Sequence Listing.xml, created on Jul. 18, 2023, and is 2,018,909 bytes in size.

### TECHNICAL FIELD

The present disclosure relates to the technical field of Nicotiana tabacum (N. tabacum) breeding, particularly to a Pseudomonas syringae pv. tabaci-resistant short WZ introgression segment, use, and screening method thereof.

### BACKGROUND

Pseudomonas syringae pv. tabaci is a major disease in N. tabacum, and the cultivation of a Pseudomonas syringae pv. tabaci-resistant variety is the most fundamental and economical means for preventing and controlling Pseudomonas syringae pv. tabaci. A promoted Pseudomonas syringae pv. tabaci-resistant variety needs to have high resistance and no yield and agronomic trait disadvantages.

At present, the resistance of a Pseudomonas syringae pv. tabaci-resistant flue-cured tobacco variety is mainly derived from Nicotiana rustica (N. rustica) var. Brasilea (wild-type (WT) N. tabacum). N. rustica cv. Brasilia (coding: W146) is hybridized with a plant of the tetraploid N. tabacum cv. Kutsaga 51, and resulting F₁ plants are a Pseudomonas syringae pv. tabaci-resistant strain No. 1. F₁ is hybridized with N. tabacum cv. Kutsaga 51 to obtain a segregation population, and a Pseudomonas syringae pv. tabaci-resistant individual is screened out through inoculation. The Pseudomonas syringae pv. tabaci-resistant individual is self-crossed for 2 generations to obtain three individuals with chromosome stability, and the three individuals are further self-crossed for 2 generations to obtain a Pseudomonas syringae pv. tabaci-resistant strain with promising breeding prospects. The yield and quality of the Pseudomonas syringae pv. tabaci-resistant strain are lower than those of a control standard variety and has the adverse trait of lodging tendency inherited from N. rustica (Ternouth et al., 1988). The inheritance of Pseudomonas syringae pv. tabaci resistance in flue-cured tobacco derived from N. rustica is controlled by a dominant single locus (Woodend, et al., 1992). The transfer of Pseudomonas syringae pv. tabaci resistance by cross-breeding refers to the transfer of a Pseudomonas syringae pv. tabaci-resistant chromosome segment of N. rustica (referred to as WZ introgression segment).

Root systems of near-isogenic lines (NILs) with or without a long WZ introgression segment are subjected to RNA sequencing analysis. SNPs, INDELs, and differentially-expressed genes among NILs are analyzed. Analysis results show that the long WZ introgression segment replaces an interval of approximately 65 Mb on chromosome 19 of N. tabacum, which indirectly indicates that the long WZ introgression segment has a size of about 65 Mb (Shi, et al., 2019).

Four possible mechanisms by which an introgression segment in N. tabacum from the WT variety leads to adverse traits include linkage drag, pleiotropy, position effects, and replacement of a chromosome segment carrying a superior gene. When any one of the above four mechanisms works, the introgression segment will bring adverse traits to a recipient parent (Chaplin and Mann, 1978). For example, it has also been reported that flue-cured tobacco with a long N introgression segment derived from Nicotiana glutinosa (N. glutinosa) has a yield reduced by 148 kg/ha on average. Flu-cured tobacco leaves have a light color, greyish at a leaf base (Chaplin et al., 1961), and high nicotine and total nitrogen (TN) contents (Lewis et al., 2007).

Studies have shown that long WZ introgression segments in flue-cured tobacco are not negatively correlated with its yield, output value, grade index, total plant alkaloid content, and reducing sugar content (Drake, et al., 2015). However, the introduction of a chromosome segment of up to 65 Mb from N. rustica into flue-cured tobacco may bring other adverse traits. It is expected to obtain a breeding line with excellent comprehensive traits by acquiring a Pseudomonas syringae pv. tabaci-resistant short WZ introgression segment and introducing the short WZ introgression segment into a flue-cured tobacco plant.

It is assumed that the linkage drag comes from non-target gene components linked to disease resistance on the long WZ introgression segment (referred to as drag gene components). The reduction of the non-target gene components linked to disease resistance on the long WZ introgression segment is expected to reduce the linkage drag of the long WZ introgression segment. However, the long WZ introgression segment has low homology with chromosomes of N. tabacum, and thus, the long WZ. introgression segment can hardly undergo a chromosome exchange with N. tabacum. Even if a chromosome exchange occurs, a conventional breeding technique cannot screen out a chromosome-exchanged plant with a short WZ introgression segment from a breeding population because a drag phenotype is a quantitative trait that is susceptible to environmental conditions and can hardly be screened out during early breeding. The acquisition of a plant with a short WZ introgression segment is expected to reduce drag gene components.

### SUMMARY

A first objective of the present disclosure is to provide a Pseudomonas syringae pv. tabaci-resistant short WZ introgression segment. Compared with the long WZ introgression segment, the reduction of non-target gene components linked to disease resistance on the long WZ introgression segment is expected to reduce the linkage drag of the long WZ introgression segment, which has promising application prospects.

A second objective of the present disclosure is to provide the use of the short WZ introgression segment described above in an N. tabacum plant. An N. tabacum plant with the short WZ introgression segment has Pseudomonas syringae pv. tabaci resistance and a lack of drag gene components linked to disease resistance, which can reduce the linkage drag of the WZ introgression segment; thus, the short WZ introgression segment can be used for selective breeding of a Pseudomonas syringae pv. tabaci-resistant N. tabacum variety without obvious yield and quality disadvantages.

A third objective of the present disclosure is to provide a screening method for an N. tabacum plant with the short WZ segment, which is easy to implement, can quickly map a position of a non-target gene segment, and can be used for accurate screening of an N. tabacum plant with the short WZ segment.

Embodiments of the present disclosure are implemented as follows:

A Pseudomonas syringae pv. tabaci-resistant short WZ introgression segment is provided. Compared with a long WZ introgression segment, the short WZ introgression segment is reduced by drag gene components of at least 500 Kb. The drag gene components include a sequence shown in SEQ ID NO: 1 and/or a sequence shown in SEQ ID NO: 2, and a gene segment adjacent to the sequence shown in SEQ ID NO: 1 and/or the sequence shown in SEQ ID NO: 2.

Use of the Pseudomonas syringae pv. tabaci-resistant short WZ introgression segment described above in an N. tabacum plant is provided.

A screening method for an N. tabacum plant with a short WZ introgression segment is provided, including:

A first parent N. tabacum plant is hybridized with a second parent N. tabacum plant to obtain an F₁ N. tabacum plant. The first parent N. tabacum plant is a homozygous N. tabacum plant with a long WZ introgression segment and has a genotype of WZWZ. The second parent N. tabacum plant is an N. tabacum plant without a long WZ introgression segment and has a genotype of wzwz. The F₁ N. tabacum plant has a genotype of WZwz.

The F₁ N. tabacum plant is self-crossed or the F₁ N. tabacum plant is back-crossed with the second parent N. tabacum plant to obtain a breeding population material with a genotype of WZwz.

Multiple primer pairs are used to test the breeding population material, and an individual plant tested as negative by at least one of the primer pairs is screened out. At least one of the primer pairs is an N. tabacum plant with a short WZ segment.

The embodiments of the present disclosure have the following beneficial effects:

An embodiment of the present disclosure provides a Pseudomonas syringae pv. tabaci-resistant short WZ introgression segment and use thereof. Compared with a long WZ introgression segment, the short WZ introgression segment is reduced by a drag gene of at least 500 Kb. The short WZ introgression segment can be used for selective breeding of a Pseudomonas syringae pv. tabaci-resistant N. tabacum variety without obvious yield and quality disadvantages, which has promising application prospects. An embodiment of the present disclosure also provides a simple screening method for an N. tabacum plant with the short WZ segment, where a homozygous N. tabacum plant with a long WZ introgression segment is hybridized with an N. tabacum plant without a long WZ introgression segment to quickly and efficiently obtain an N. tabacum plant with the short WZ segment, which provides an experimental basis for the acquisition of the Pseudomonas syringae pv. tabaci-resistant short WZ introgression segment.

### DETAILED DESCRIPTION OF THE EMBODIMENTS

To make the objectives, technical solutions, and advantages of the embodiments of the present disclosure clear, the technical solutions in the embodiments of the present disclosure will be described clearly and completely below. If no specific conditions are specified in the embodiments, the embodiments will be implemented under conventional conditions or the conditions recommended by the manufacturer. All of the used reagents or instruments are conventional commercially-available products if the manufacturer has not been identified

The Pseudomonas syringae pv. tabaci-resistant short WZ introgression segment, the use, and the screening method thereof in an embodiment of the present disclosure are specifically described below.

An embodiment of the present disclosure provides a Pseudomonas syringae pv. tabaci-resistant short WZ introgression segment. Compared with a long WZ introgression segment, the short WZ introgression segment is reduced by a drag gene component of at least 500 Kb. The drag gene component includes a sequence shown in SEQ ID NO: 1 and/or a sequence shown in SEQ ID NO: 2, and a gene segment adjacent to the sequence shown in SEQ ID NO: 1 and/or the sequence shown in SEQ ID NO: 2.

In the context of the present disclosure, the term “WZ introgression segment” refers to a chromosome segment with Pseudomonas syringae pv. tabaci resistance from WT N. rustica.

The term “short WZ introgression segment” refers to a DNA segment obtained by partially or completely deleting a non-target gene component (referred to as a drag gene component) linked to disease resistance on the WZ introgression segment and retaining disease resistance such as Pseudomonas syringae pv. tabaci.

The term “drag gene component” refers to a gene component on the WZ introgression segment that includes a drag gene but does not have resistance for a target disease such as Pseudomonas syringae pv. tabaci.

Studies have shown that the long WZ introgression segment replaces an interval of about 65 Mb on chromosome 19 of N. tabacum, which indirectly indicates that the long WZ introgression segment has a size of about 65 Mb (Shi, et al., 2019. Supplemental Table S1. Primer sequences used for KASP or CAPS SNP marker genotyping for the presence/absence of the introgressed). The genomic diversity between the long WZ introgression segment and chromosome 19 of N. tabacum will significantly limit a chromosome exchange, resulting in a low possibility of recombination between the long WZ introgression segment and N. tabacum. The introduction of a chromosome segment of up to 65 Mb from N. rustica into flue-cured tobacco is very likely to bring other adverse traits for an N. tabacum plant. In an embodiment of the present disclosure, a drag gene component of at least 500 Kb is reduced from the original WZ introgression segment to obtain a Pseudomonas syringae pv. tabaci-resistant short Wz introgression segment, and the introduction of the short Wz introgression segment into a flue-cured tobacco plant is expected to reduce the linkage drag of the long WZ introgression segment, thereby acquiring a breeding line with excellent comprehensive traits.

The short WZ introgression segment is tested as positive for a KASP-10.6 primer pair and a KASP-29.9 primer pair, and the short WZ introgression segment is tested as negative for a KASP-5.1 primer pair and/or a KASP-42.3 primer pair. The above sequences are shown in Table 1.

TABLE 1
Primer pair sequences

| Primer pair | | | |
|---|---|---|---|
| name | Primer name | Primer sequence | Remarks |
| KASP-5.1 | KASP-5.1-F | GAAGGTGACCAAGTTCATGCTCGTTTACTGGC | SEQ ID NO: 3 |
| | | GCCTGATC | |
| | KASP-5.1-H | GAAGGTCGGAGTCAACGGATTGTTTACTGGCG | SEQ ID NO: 4 |
| | | CCTGAGT | |
| | KASP-5.1-CR | CTCCGTCTACACTGACTACCT | SEQ ID NO: 5 |
| KASP-10.6 | KASP-10.6-F | GAAGGTGACCAAGTTCATGCTTCAGTGGCAAA | SEQ ID NO: 6 |
| | | AGCTGCAAG | |
| | KASP-10.6-H | GAAGGTCGGAGTCAACGGATTTCAGTGGCAA | SEQ ID NO: 7 |
| | | AAGCTGCTAA | |
| | KASP-10.6-CR | GCCACACAGGATCTCTCATTGATTG | SEQ ID NO: 8 |
| KASP-29.9 | KASP-29.9-F | GAAGGTGACCAAGTTCATGCTCACTGATTCAG | SEQ ID NO: 9 |
| | | ATCTTGAGGGAT | |
| | KASP-29.9-H | GAAGGTCGGAGTCAACGGATTACACTGATTCA | SEQ ID NO: 10 |
| | | GATCTTGAGGAAG | |
| | KASP-29.9-CR | GTTTTCCCCACACTCAGGT | SEQ ID NO: 11 |
| KASP-42.3 | KASP-42.3-F | GAAGGTGACCAAGTTCATGCTAGATACCTTTC | SEQ ID NO: 12 |
| | | CGAACTCCGG | |
| | KASP-42.3-H | GAAGGTCGGAGTCAACGGATTGAGATACCTTT | SEQ ID NO: 13 |
| | | CCGAACTCCCA | |
| | KASP-42.3-CR | GGAGAGGTCCAAAGTTACTAAATCG | SEQ ID NO: 14 |

Notes:
F: FAM-tag, H: HEX-tag, CR: Commen Reverse

The short WZ introgression segment is tested as positive for a KASP-10.6 primer pair and a KASP-29.9 primer pair, indicating that gene sequences corresponding to the KASP-10.6 primer pair and the KASP-29.9 primer pair are retained in the short WZ introgression segment, which are target gene sequences associated with Pseudomonas syringae pv. tabaci resistance. The short WZ introgression segment is tested as negative for a KASP-5.1 primer pair and/or a KASP-42.3 primer pair, indicating that gene sequences corresponding to the KASP-5.1 primer pair and/or the KASP-42.3 primer pair are deleted in the short WZ introgression segment, which are non-target gene sequences, namely, drag genes.

Further, those skilled in the art should understand that 1 to 30 bases may be added to a 5′ or 3′ terminus of each of the sequences shown in SEQ ID NOS: 3-14, and a type of added base can be determined according to a type of a base in a region matching each of SEQ ID NOS: 3-14 on the N. tabacum genome DNA and a principle of base pairing, and a resulting primer pair is basically the same as an amplification product for each of SEQ ID NOS: 3-14 (a DNA sequence between upstream and downstream primers). Thus, primer pairs obtained by adding 1 to 30 bases to a 5′ or 3′ terminus of each of SEQ ID NOS: 3-14 that can amplify substantially the same DNA segments are included in the primer pairs of the present disclosure. In a specific embodiment of the present disclosure, the primer pairs of the present disclosure are preferably sequences shown in SEQ ID NOS: 3-14.

Further, the short WZ introgression segment is obtained by chromosome exchange, genome editing, chemical mutagenesis, or physical mutagenesis. There is also a crossbreeding method, where a WZwz population material is bred, and then a chromosome-exchanged plant is screened out. The chemical or physical mutagenesis method or the biotechnological genome editing method is used to screen out a mutant in which the sequence of the drag gene with a sequence shown in SEQ ID NO: 1 and/or SEQ ID NO: 2 is partially or completely deleted. The chemical mutagenesis method includes treatment by a mutagenic agent such as sodium azide, ethidium bromide, ethyl methanesulfonate (EMS), or the like. The physical mutagenesis method includes X-ray radiation, γ-ray radiation, fast neutron radiation, heavy ion radiation, and ultraviolet (UV) radiation. The biotechnological genome editing method includes the use of a CRISPR/Cas9 technology, a zinc-finger nuclease (ZFN) technology, or a transcription activator-like effector nuclease (TALEN) technology to knock out a part or all of the sequences of the drag gene shown in SEQ ID NO: 1 and/or SEQ ID NO: 2. The partial sequence has an appropriate length, which is not particularly limited, and may be 10 Kb, 100 Kb, 500 Kb, or 1,000 Kb, for example.

An embodiment of the present disclosure also provides the use of the Pseudomonas syringae pv. tabaci-resistant short WZ introgression segment described above in an N. tabacum plant.

Those skilled in the art should understand that the short WZ introgression segment can be used for common N. tabacum varieties, including, but not limited to, K326, Yunyan 87, Yunyan 97, Yunyan 85, Yunyan 116, Yunyan 121, NC89, Zhongyan 100, Honghuadajinyuan, and Cuibi No. 1.

An embodiment of the present disclosure also provides a screening method for an N. tabacum plant with a short WZ segment, including:

SI. A first parent N. tabacum plant is hybridized with a second parent N. tabacum plant to obtain a F₁ N. tabacum plant.

The first parent N. tabacum plant is a homozygous N. tabacum plant with a long WZ introgression segment and has a genotype of WZWZ, including, but not limited to, flue-cured tobacco, burley tobacco, air-cured tobacco, aromatic tobacco, and cigar tobacco. The second parent N. tabacum plant is an N. tabacum plant without a long WZ introgression segment and has a genotype of wzwz, including, but not limited to, flue-cured tobacco, burley tobacco, air-cured tobacco, aromatic tobacco, and cigar tobacco. Phenotypic traits of the N. tabacum with the genotype of wzwz include disease resistance, high yield, high-grade index, easy baking, leaf quality, plant height, maturity characteristics of anywhere between early maturity and late maturity, and plant leaf number from medium to high. The N. tabacum with the genotype of wzwz preferably includes, but is not limited to, K326, Yunyan 87, Yunyan 97, Yunyan 85, NC89, Zhongyan 100, Honghuadajinyuan, and Cuibi No. 1. The F₁ N. tabacum plant obtained by the hybridization has a genotype of WZwz.

The screening method for an N. tabacum plant with a short WZ segment provided in the embodiment of the present disclosure further includes:

S2. The F₁ N. tabacum plant is self-crossed or back-crossed with the second parent N. tabacum plant to obtain a breeding population material with a genotype of WZwz.

The population material includes a breeding population material with a genotype of WZwz and is preferably a backcrossing population material BC₁F₁, BC₂F₁, BC₃F₁, BC₄F₁, BC₅F₁ . . . BCₙF₁, where n represents a number of generations of backcrossing, which may be 6, 7, 8, 9, 10, or 15. The population material also includes another population material, such as a self-crossing population material F₂ with a genotype of WZwz.

S3. Multiple primer pairs are used to test the breeding population material, and an individual plant tested as negative for at least one of the primer pairs is screened out, which is an N. tabacum plant with a short WZ segment.

The selected primer pairs are molecular markers of the WZ gene or molecular markers closely linked to the WZ gene. A negative test result indicates that gene sequences corresponding to the primer pair are deleted, and a positive test result indicates that gene sequences corresponding to the primer pair are retained. According to molecular marker test results, short WZ introgression segments with gene sequence deletions at different locations can be obtained.

According to molecular marker test results, a disease-resistant phenotype selection method is also required to further map a location of a drag gene, including:

S4. Pseudomonas syringae is inoculated into different N. tabacum seedlings of the individual plant, and a Pseudomonas syringae pv. tabaci-resistant plant is screened out. According to the test results of the primer pairs for the Pseudomonas syringae pv. tabaci-resistant plant, a primer pair leading to a negative test result is selected, and sequences corresponding to the primer pair are denoted as marker sequences.

The marker sequences are only a small part of the sequence of the drag gene, which can only help locate a location of the drag gene; and genome resequencing is further required to obtain an accurate sequence of the drag gene, including:

S5. DNA of the Pseudomonas syringae pv. tabaci-resistant plant is extracted and subjected to genome resequencing, and a sequencing result is compared with the first parent N. tabacum plant and the second parent N. tabacum plant to determine a length range of a drag gene with the marker sequences.

Similarly, with the above method, a Pseudomonas syringae pv. tabaci-resistant plant can also be screened out. According to the test results of the primer pairs for the Pseudomonas syringae pv. tabaci-resistant plant, a primer pair leading to a negative test result is selected, and sequences corresponding to the primer pair are denoted as marker sequences. In this case, the primer pair leading to a negative test result marks an approximate location of the Pseudomonas syringae pv. tabaci-resistant target gene, which lays a foundation for the next step to achieve the accurate localization of the Pseudomonas syringae pv. tabaci-resistant gene.

After the influence of gene sequences corresponding to each primer pair on a disease resistance phenotype of an N. tabacum plant is determined, a Pseudomonas syringae pv. tabaci-resistant N. tabacum plant with a short WZ introgression segment can be quickly screened out through molecular marker test results.

Optionally, the multiple primer pairs include a KASP-5.1 primer pair, a KASP-10.6 primer pair, a KASP-29.9 primer pair, and a KASP-42.3 primer pair. The combination of the above four primer pairs enables the rapid screening of a Pseudomonas syringae pv. tabaci-resistant N. tabacum plant with a short WZ introgression segment. Specifically, when tested as positive by a KASP-10.6 primer pair and a KASP-29.9 primer pair and is tested as negative by a KASP-5.1 primer pair and/or a KASP-42.3 primer pair, an individual plant exhibits prominent Pseudomonas syringae pv. tabaci resistance. On the contrary, when tested as negative by a KASP-10.6 primer pair or a KASP-29.9 primer pair, an individual plant exhibits Pseudomonas syringae pv. tabaci susceptibility.

The features and properties of the present disclosure are further described in detail below in conjunction with the examples.

### EXAMPLE 1

In this example, a preparation and DNA extraction method for a plant material was provided, including:

A first parent plant (T6, genotype: WZWZ, Pseudomonas syringae pv. tabaci resistance) and a second parent plant (Y87, genotype: wzwz, Pseudomonas syringae pv. tabaci susceptibility) were cultivated and hybridized to obtain an F₁ seed (genotype: WZwz).

The F₁ plant was cultivated, pollen was collected from the F₁ plant and pollinated on emasculated Y87 flowers at a flowering stage, and a BC₁F₁ seed was harvested (genotype: WZwz: wzwz=1:1).

The BC₁F₁ seed was sown, tobacco seedlings were numbered, and leaves were taken for DNA extraction. DNA was extracted by the CTAB method or the DNeasy plant 96 Plant Kit (QIAGEN).

### EXAMPLE 2

In this example, a screening method for a plant with a short WZ introgression segment was provided, including:

KASP genotyping was conducted. A Pseudomonas syringae pv. tabaci-susceptible N. tabacum variety Y87 served as a Pseudomonas syringae pv. tabaci susceptibility control A Pseudomonas syringae pv. tabaci-resistant N. tabacum variety T6 with a long WZ introgression segment served as a Pseudomonas syringae pv. tabaci resistance control. A KASP-5.1 primer pair, a KASP-10.6 primer pair, a KASP-29.9 primer pair, and a KASP-42.3 primer pair served as primers. DNA of N. tabacum to be tested, DNA of Pseudomonas syringae pv. tabaci-susceptible N. tabacum control, and DNA of Pseudomonas syringae pv. tabaci-resistant N. tabacum control served as templates.

### KASP Genotyping Method

PCR system: 2×KASP Master mix: 5 μl, primer pair: 0.7 μl, and DNA: 5 μl.

PCR conditions: 94° C., 15 min; (94° C., 20 S; 61° C. to 55° C., 60 S) 10 cycles (decreasing by 0.6° C. per cycle); (94° C., 20 S; 55° C., 60 S) 26 cycles. If fully clear genotype clustering information was not available after the initial KASP thermal cycling, 3 cycles of (94° C., 20 S; 57° C., 60 S) were added at a time until clear genotype clustering information was available. After the thermal cycling was completed, a reaction plate was read using a microplate reader FLUOstar Omega SNP with an FRET function. The data were imported into the genotype clustering software package KlusterCaller™ (LGC) (www.lgcgroup.com/software) and subjected to graphing and genotype clustering analysis.

The primer pairs were a KASP-5.1 primer pair, a KASP-10.6 primer pair, a KASP-29.9 primer pair, and a KASP-42.3 primer pair. The reagents used were purchased from LGC.

An N. tabacum plant with a genotype tested as Y87 by a KASP-5.1 primer pair, an N. tabacum plant with a genotype tested as heterozygous T6 by a KASP-10.6 or KASP-29.9 primer pair, an N. tabacum plant with a genotype tested as Y87 by a KASP-42.3 primer pair, and an N. tabacum plant with a genotype tested as heterozygous T6 by a KASP-10.6 or KASP-29.9 primer pair each were screened out.

T6 was flue-cured tobacco with a long WZ introgression segment (genotype: WZWZ), and Y87 was flue-cured tobacco without a WZ introgression segment (genotype: wzwz). KASP-5.1, KASP-10.6, KASP-29.9, and KASP-42.3 primer pairs were used for test. If an individual plant was tested by the KASP-10.6 primer pair and/or the KASP-29.9 primer pair to have a heterozygous T6 genotype and tested by the KASP-5.1 primer pair to have a Y87 genotype, indicating that, in the individual plant, a chromosome segment where the KASP-5.1 primer pair was located (namely, a sequence shown in SEQ ID NO: 1) was deleted. If an individual plant was tested by the KASP-10.6 primer pair and/or the KASP-29.9 primer pair to have a heterozygous T6 genotype and tested by the KASP-42.3 primer pair to have a Y87 genotype, indicating that, in the individual plant, a chromosome segment where the KASP-42.3 primer pair was located (namely, a sequence shown in SEQ ID NO: 2) was deleted. A plant with the deletion of the chromosome segment where the KASP-5.1 primer pair was located alone, a plant with the deletion of the chromosome segment where the KASP-42.3 primer pair was located alone, or a plant with the deletion of both the chromosome segments where the KASP-5.1/KASP-42.3 primer pairs were respectively located was a plant with a short WZ introgression segment. Results showed that 9 plants each with a short WZ introgression segment were screened out from 4,464 BC₁F₁ plants of Y87×T6 (as shown in Table 2). These 9 plants were chromosome-exchanged plants each carrying a short WZ introgression segment.

TABLE 2
Individual plants each with a short WZ introgression segment

| | Genotype | Genotype | Genotype | Genotype | |
|---|---|---|---|---|---|
| | detected | detected | detected | detected | |
| Individual | by the | by the | by the | by the | |
| plant | KASP-5.1 | KASP-10.6 | KASP-29.9 | KASP-42.3 | |
| DNA No. | primer pair | primer pair | primer pair | primer pair | Remarks |
| T6 | Y | Y | Y | Y | Homozygous long WZ segment |
| R0 | XY | XY | XY | XY | Heterozygous long WZ segment |
| 52-8E | X | XY | XY | XY | Deletion of a sequence |
| | | | | | corresponding to KASP-5.1 |
| 42-4F | X | XY | XY | XY | Deletion of a sequence |
| | | | | | corresponding to KASP-5.1 |
| 20-11E | X | XY | XY | XY | Deletion of a sequence |
| | | | | | corresponding to KASP-5.1 |
| 49-12B | XY | XY | XY | X | Deletion of a sequence |
| | | | | | corresponding to KASP-42.3 |
| 23-2F | X | X | XY | XY | Deletion of a sequence |
| | | | | | corresponding to KASP-5.1 |
| 53-5F | X | X | XY | XY | Deletion of sequences |
| | | | | | corresponding to KASP-5.1/10.6 |
| 22-1B | X | X | X | XY | Deletion of sequences |
| | | | | | corresponding to KASP-5.1/10.6 |
| 33-4F | X | X | X | XY | Deletion of sequences |
| | | | | | corresponding to |
| | | | | | KASP-5.1/10.6/29.9 |
| 44-6B | X | X | X | XY | Deletion of sequences |
| | | | | | corresponding to |
| | | | | | KASP-5.1/10.6/29.9 |
| Y87 | X | X | X | X | No WZ introgression segment |

Notes:
Y represents a homozygous T6 genotype (WZWZ), X represents a homozygous Y87 genotype (wzwz), and XY represents a heterozygous T6 genotype.

### EXAMPLE 3

In this example, a method for screening a Pseudomonas syringae pv. tabaci-resistant plant from the plants each with a short WZ introgression segment was provided, including:

### 1. Preparation of a Pseudomonas syringae Inoculum

A Pseudomonas syringae strain 59 # was taken from a 20% glycerol tube stored at −20° C. Nutrient medium formula of 3 g/L beef extract, 5 g/L peptone, 5 g/L glucose, and 15 g/L agar were mixed and diluted to 1,000 mL, adjusted to a PH of 7.3, and sterilized at 120° C. for 20 min. The strain was activated on the nutrient medium at 28° C. for 48 h. Pseudomonas syringae colonies with excellent vitality were picked, streaked on a Petri dish with the nutrient medium, and cultivated at 28° C. for 48 h. The resulting mosses were scraped off, put into sterile water, and stirred to obtain a bacterial solution, and a concentration of the bacterial solution was adjusted to an OD₆₀₀ value of 1.0.

### 2. Inoculation Method

3 to 4-leaf tobacco seedlings were planted in pots that each were filled with an N. tabacum seedling substrate and had a diameter of 21 cm. When N. tabacum seedlings each had 5 to 6 new leaves, a needle was used to puncture the 3rd to 5th leaves from top to bottom with three points at each of left and right sides of each leaf. The bacterial solution was mist-sprayed on the needle-punctured points with a small handheld watering can. The temperature of an air conditioner was set to 28° C., and the seedlings were cultivated in 16 h light/8 h dark every day. The seedlings were humidified in the first 5 days after inoculation, that is, a humidity was adjusted with a humidifier to 80% or a handheld sprayer was used to spray 6 times every day, and the humidification was stopped 5 days after inoculation.

### 3. Pseudomonas syringae pv. Tabaci Spot Size Measurement and Resistance Evaluation

7 to 14 days after inoculation, a vernier caliper was used to measure distances of an outer edge of a yellow halo of a Pseudomonas syringae pv. tabaci spot from maximized and minimized positions of an edge of a needle pore at an inoculation site, and an average value was taken as a diameter of the spot at the inoculation site. Spot diameters at all inoculation sites of each N. tabacum plant were measured, and an average spot diameter (mm) of inoculated leaves of each N. tabacum plant was calculated. Through comparison with average spot diameters of the disease-resistant and disease-susceptible control plants, the resistance of a test variety was determined. When a spot diameter was less than or equal to the average spot diameter of the disease-resistant control plant, a test variety was resistant. When a spot diameter was equal to or greater than the average spot diameter of the disease-susceptible control plant, a test variety was susceptible. Results are shown in Table 3.

TABLE 3
Individual plants each with a short WZ introgression segment
and Pseudomonas syringae pv. tabaci resistance thereof

| | | Genotype | Genotype | Genotype | Genotype | | Pseudomonas |
|---|---|---|---|---|---|---|---|
| | Individual | detected by the | detected by the | detected by the | detected by the | Spot | syringae pv. |
| Individual | plant | KASP-5.1 | KASP-10.6 | KASP-29.9 | KASP-42.3 | diameter | tabaci |
| plant No. | DNA No. | primer pair | primer pair | primer pair | primer pair | (mm) | resistance |
| T6 | T6 | Y | Y | Y | Y | 0 | Resistance |
| R0 | R0 | XY | XY | XY | XY | 0 | Resistance |
| R1 | 52-8E | X | XY | XY | XY | 0 | Resistance |
| R2 | 42-4F | X | XY | XY | XY | 0.22 | Resistance |
| R3 | 20-11E | X | XY | XY | XY | 0.11 | Resistance |
| R4 | 49-12B | XY | XY | XY | X | 0 | Resistance |
| S2 | 23-2F | X | X | XY | XY | 2.74 | Susceptibility |
| S6 | 53-5F | X | X | XY | XY | 1.28 | Susceptibility |
| S1 | 22-1B | X | X | X | XY | 5.55 | Susceptibility |
| S4 | 33-4F | X | X | X | XY | 1.31 | Susceptibility |
| S5 | 44-6B | X | X | X | XY | 1.34 | Susceptibility |
| Y87 | Y87 | X | X | X | X | 3.64 | Susceptibility |

Notes:
Y represents a homozygous T6 genotype, X represents a homozygous Y87 genotype, and XY represents a heterozygous T6 genotype.

It can be seen from the identified Pseudomonas syringae pv. tabaci resistance of the plants each with a short WZ introgression segment obtained in Example 2 in Table 3 that 4 individual plants each with a short WZ introgression segment (numbered R1, R2, R3, and R4) are resistant to Pseudomonas syringae pv. tabaci. In combination with the results in Table 2, it can be known that a sequence corresponding to KASP-5.1 is deleted in R1 to R3, and a sequence corresponding to KASP-42.3 is deleted in R4. In each of these 4 individual plants each with a Pseudomonas syringae pv. tabaci-resistant short WZ introgression segment, the non-target gene component is partly deleted, and the Pseudomonas syringae pv. tabaci-resistant target gene component is retained. The size of the non-target gene component deleted is analyzed.

In addition, 5 individual plants each with a short WZ introgression segment (numbered S1, S2, S4, S5, and S6) are susceptible to Pseudomonas syringae pv. tabaci. In combination with the results in Table 2, it can be known that sequences corresponding to KASP-5.1/10.6 are deleted in S2 and S6, and sequences corresponding to KASP-5.1/10.6/29.9 are deleted in S1, S4, and S5. In each of these 5 individual plants, the Pseudomonas syringae pv. tabaci-resistant target gene component is deleted.

### EXAMPLE 4

In this example, a method for detecting a number of the reduced non-target gene components linked to disease resistance was provided, including:

Leaves were collected from the 9 individual plants each with a short WZ introgression segment obtained in Example 2, and DNA was extracted and sent to Huada Gene for genome resequencing and bioinformatics analysis. A BGISEQ-500 platform was used for sequencing with a sequencing depth of 10×. Resequencing data was subjected to SNP variation analysis, each chromosome of each offspring sample slid with 50 kb as a bin window, and an SNP site of each offspring was compared with the SNP sites of the two parents. If an SNP site of an offspring sample was the same as the parent T6, the offspring sample was marked as p1. If an SNP site of an offspring sample was the same as the parent Y87, the offspring sample was marked as p2. If an SNP site of an offspring sample was deleted or heterozygous, the offspring sample was marked as a miss. SNP Maker information of each offspring sample of the population was obtained in the bin. A sliding window (with 15 markers per window) was used to find accurate recombination points, and a physical recombination map was plotted for each chromosome of the 9 individual plants each with a short WZ introgression segment.

According to the physical recombination maps of the disease-resistant and disease-susceptible individual plants each with a short WZ introgression segment, genome sequences of 500 Kb at two sides of SNP where the KASP-5.1 and KASP-42.3 primer pairs were located respectively were determined, namely, sequences shown in SEQ ID NO: 1 and SEQ ID NO: 2 and a gene segment adjacent to a sequence shown in SEQ ID NO: 1 and/or a sequence shown in SEQ ID NO: 2.

In summary, an embodiment of the present disclosure provides a Pseudomonas syringae pv. tabaci-resistant short WZ introgression segment and use thereof. Compared with a long WZ introgression segment, the short WZ introgression segment is reduced by a drag gene of at least 500 Kb. The short WZ introgression segment can be used for selective breeding of a Pseudomonas syringae pv. tabaci-resistant N. tabacum variety without obvious yield and quality disadvantages, which has promising application prospects. An embodiment of the present disclosure also provides a simple screening method for an N. tabacum plant with the short WZ segment, where a homozygous N. tabacum plant with a long WZ introgression segment is hybridized with an N. tabacum plant without a long WZ introgression segment to quickly and efficiently obtain an N. tabacum plant with the short WZ segment, which provides an experimental basis for the acquisition of the Pseudomonas syringae pv. tabaci-resistant short WZ introgression segment.

The above are merely preferred examples of the present disclosure and are not intended to limit the present disclosure, and various changes and modifications may be made by those skilled in the art to the present disclosure. Any modifications, equivalent substitutions, improvements, and the like made within the spirit and principle of the present disclosure should be included within the protection scope of the present disclosure.

What is claimed is:

1. A Pseudomonas syringae pv. tabaci-resistant short WZ introgression segment, wherein compared with a long WZ introgression segment, the Pseudomonas syringae pv. tabaci-resistant short WZ introgression segment is reduced by a drag gene component of at least 500 Kb; and the drag gene component comprises: a sequence shown in SEQ ID NO: 1 and/or a sequence shown in SEQ ID NO: 2, and a gene segment adjacent to the sequence shown in the SEQ ID NO: 1 and/or the sequence shown in the SEQ ID NO: 2.

2. The Pseudomonas syringae pv. tabaci-resistant short WZ introgression segment according to claim 1, wherein the Pseudomonas syringae pv. tabaci-resistant short WZ introgression segment is tested as positive for a KASP-10.6 primer pair and a KASP-29.9 primer pair; and the Pseudomonas syringae pv. tabaci-resistant short WZ introgression segment is tested as negative for a KASP-5.1 primer pair and/or a KASP-42.3 primer pair.

3. The Pseudomonas syringae pv. tabaci-resistant short WZ introgression segment according to claim 2, wherein the Pseudomonas syringae pv. tabaci-resistant short WZ introgression segment is obtained by a chromosome exchange, a genome editing, a chemical mutagenesis, or a physical mutagenesis.

4. A method of an use of the Pseudomonas syringae pv. tabaci-resistant short WZ introgression segment according to claim 1 in a Nicotiana tabacum (N. tabacum) plant.

5. A screening method for an N. tabacum plant with a short WZ segment, comprising:
hybridizing a first parent N. tabacum plant with a second parent N. tabacum plant to obtain a F₁ N. tabacum plant, wherein the first parent N. tabacum plant is a homozygous N. tabacum plant with a long WZ introgression segment and has a genotype of WZWZ; the second parent N. tabacum plant is an N. tabacum plant without the long WZ introgression segment and has a genotype of wzwz; and the F₁ N. tabacum plant has a genotype of WZwz;
self-crossing the F₁ N. tabacum plant or back-crossing the F₁ N. tabacum plant with the second parent N. tabacum plant to obtain a breeding population material with the genotype of WZwz; and
using multiple primer pairs to test the breeding population material, and screening out an individual plant tested as negative by at least one of the multiple primer pairs, wherein the individual plant is an N. tabacum plant with the short WZ segment.

6. The screening method according to claim 5, wherein the first parent N. tabacum plant is a Pseudomonas syringae pv. tabaci-resistant N. tabacum plant T6, and the second parent N. tabacum plant comprises any one selected from the group consisting of K326, Yunyan 87. Yunyan 97, Yunyan 85, NC89, Zhongyan 100, Honghuadajinyuan, and Cuibi No. 1.

7. The screening method according to claim 5, further comprising:
inoculating Pseudomonas syringae into different N. tabacum seedlings of the individual plant, and screening out a Pseudomonas syringae pv. tabaci-resistant plant; and according to test results of the multiple primer pairs for the Pseudomonas syringae pv. tabaci-resistant plant, selecting a primer pair leading to a negative test result, and denoting sequences corresponding to the primer pair as marker sequences.

8. The screening method according to claim 7, further comprising:
extracting a DNA of the Pseudomonas syringae pv. tabaci-resistant plant, and subjecting the DNA to a genome resequencing; and
comparing a sequencing result with the first parent N. tabacum plant and the second parent N. tabacum plant to determine a length range of a drag gene with the marker sequences.

9. The screening method according to claim 5, wherein the multiple primer pairs comprise a KASP-5.1 primer pair, a KASP-10.6 primer pair, a KASP-29.9 primer pair, and a KASP-42.3 primer pair.

10. The screening method according to claim 9, wherein the individual plant is tested as positive for the KASP-10.6 primer pair and the KASP-29.9 primer pair; and the individual plant is tested as negative for the KASP-5.1 primer pair and/or the KASP-42.3 primer pair.

11. The method of the use of the Pseudomonas syringae pv. tabaci-resistant short WZ introgression segment according to claim 4, wherein the Pseudomonas syringae pv. tabaci-resistant short WZ introgression segment is tested as positive for a KASP-10.6 primer pair and a KASP-29.9 primer pair; and the Pseudomonas syringae pv. tabaci-resistant short WZ introgression segment is tested as negative for a KASP-5.1 primer pair and/or a KASP-42.3 primer pair.

12. The method of the use of the Pseudomonas syringae pv. tabaci-resistant short WZ introgression segment according to claim 11, wherein the Pseudomonas syringae pv. tabaci-resistant short WZ introgression segment is obtained by a chromosome exchange, a genome editing, a chemical mutagenesis, or a physical mutagenesis.

13. The screening method according to claim 6, further comprising:
inoculating Pseudomonas syringae into different N. tabacum seedlings of the individual plant, and screening out a Pseudomonas syringae pv. tabaci-resistant plant; and according to test results of the multiple primer pairs for the Pseudomonas syringae pv. tabaci-resistant plant, selecting a primer pair leading to a negative test result, and denoting sequences corresponding to the primer pair as marker sequences.

14. The screening method according to claim 6, wherein the multiple primer pairs comprise a KASP-5.1 primer pair, a KASP-10.6 primer pair, a KASP-29.9 primer pair, and a KASP-42.3 primer pair.

15. The screening method according to claim 13, further comprising:
extracting a DNA of the Pseudomonas syringae pv. tabaci-resistant plant, and subjecting the DNA to a genome resequencing; and
comparing a sequencing result with the first parent N. tabacum plant and the second parent N. tabacum plant to determine a length range of a drag gene with the marker sequences.

16. The screening method according to claim 14, wherein the individual plant is tested as positive for the KASP-10.6 primer pair and the KASP-29.9 primer pair, and the individual plant is tested as negative for the KASP-5.1 primer pair and/or the KASP-42.3 primer pair.
